(19) United States
(12) Patent Application Publication
(10) Pub. No.: US 2012/0088773 A1
(43) Pub. Date: Apr. 12, 2012

(54) Circadian rhythm modulation

(76) Inventor: Ronald J. Thompson, Fort Thomas, KY (US)

(21) Appl. No.: 12/924,858

(22) Filed: Oct. 6, 2010

(51) Int. Cl. A61K 31/4985; A61P 3/00; A61K 31/522

(52) U.S. Cl. 514/250; 514/263.31

(57) ABSTRACT

A method and composition is herein described to modulate the circadian rhythm to effect summer metabolism and the metabolic syndrome. The method entails the once daily, timed ingestion of a dopamine precursor or a dopamine agonist, receptor primed with caffeine, and vitamin D₃.

### FIELD OF THE INVENTION

This invention defines the once daily, timed concurrent use of medicaments to effect summer metabolism and the metabolic syndrome.

### DEFINITIONS FOR DISCLOSURE

Agonist—a chemical substance capable of activating a receptor to induce a full or partial pharmacological response.
Bromocriptine—a pharmaceutical chemical: a dopamine agonist. Bromocriptine was FDA approved as Parlodel in 1985 for suppression of hyperprolactinemia, a cause of infertility. Parlodel 2.5 was also used to prevent postpartum breast engorgement of women who chose not to breastfeed.
Caffeine—a xanthine alkaloid with neurostimulant properties that produces such an effect by acting as a competitive inhibitor at adenosine receptors, thus blocking the inhibitory effects of adenosine. Reduction in adenosine leads increased activity levels of the neurotransmitter dopamine.
Catecholamine—amine structured neurotransmitters including: dopamine, epinephrine, and norepinephrine.
Chronobiotics—pharmaceutical chemicals employed to modulate or influence the circadian rhythm or the diurnal rhythm.
Circadian Rhythm—of, or pertaining to, rhythmic cycles recurring at approximately 24-hour intervals: the circadian biological clock.
Diabetes (Type 2)—a disease or condition where insulin does not effectively transport glucose from the bloodstream into cells: a slowly developing condition in which the body's tissues become unable to use insulin effectively.
Diurnal Rhythm—daily cycles with two reproducible, predicted events: like the opening by day and closing by night of certain flowers.
Dopamine—a neurotransmitter from the catecholamine family. Acts both as a neurotransmitter and a hormone inhibiting factor (inhibits prolactin release). Is synthesized from the amino acid L-tyrosine.
Hypothalamus-Pituitary Axis—the hypothalamus is a region of the brain diencephalon that is the regulating center for visceral functions such as: sleep cycles, body temperature, and the activity of the pituitary gland. The hypothalamus accepts external stimuli from all of the five senses; especially light via the pineal gland-optic nerve, and internal stimuli from end organs that the pituitary hormones stimulate. The pituitary gland produces hormones that are released into the bloodstream at the direction of the hypothalamus. The hypothalamus produces and releases stimulating and inhibiting hormones into the hyposeal-portal circulation that only effect an action in the specific pituitary tissues. These hypothalamus-pituitary neurotransmitters are referred to as releasing factors. The stimulatory releasing factors are: follicle stimulating hormone (FSH) releasing factor, luteinizing hormone (LH) releasing factor, thyroid hormone (TH) releasing factor, adrenocorticotropic hormone (ACTH) releasing factor, growth hormone (GH) releasing factor, and melanocyte hormone releasing factor. The inhibiting releasing factor is prolactin inhibiting releasing factor. A stimulating releasing factor for prolactin is unknown, but both thyroid hormone (TH) and growth hormone (GH) releasing factors can stimulate pituitary prolactin secretion. The hypothalamus and pituitary gland are also connected by efferent nerves, but the primary control of the pituitary gland functions are by the stimulating and inhibiting hypothalamic releasing factors. This is the hypothalamus-pituitary axis.
Insulin Resistance—is a disease/condition where a normal amount of insulin is produced from the beta cells of the pancreas, but the insulin does not function properly at the cellular level to transport glucose from the blood into those cells. Insulin resistance is associated with altered gluconeogenesis and dyslipidemia.
Metabolic Syndrome—is a pre-diabetic (type 2) state that is defined by four conditions:
   Insulin resistance
   Increased weight (increased abdominal circumference)
   Dyslipidemia (elevated cholesterol/lipid profile)
   Hypertension

The metabolic syndrome is a progressive condition that elevates a person's cardiovascular risk factors. The metabolic syndrome usually develops in the third or fourth decade of life and is directly responsible for the increasing incidence of cardiovascular disease in women more than men.
Neurotransmitter—the catecholamines are neurotransmitters. The neurotransmitters are produced in the actual nerve and upon stimulation, are released by the distal end of the nerve into the synaptic space. The neurotransmitter catecholamines: dopamine, epinephrine, and norepinephrine stimulate the proximal aspect of multiple adjoining nerves. In this architecture, the stimulated nerve can stimulate multiple nerves to amplify the single signal, all because of the neurotransmitter and the synapse.
Vitamin D₃ (cholecalciferol)—a hormone that is photosynthesized in the skin of vertebrates by the action of solar ultraviolet (UV) B radiation on 7-dehydrocholesterol. The physiologically active form of vitamin D is the D₃ form. D₁ and D₂ are precursors of vitamin D₃. Only vitamin D₁ and D₂ are available from dietary sources (CDC).

### PRIOR ART—INTELLECTUAL PROPERTY DISCUSSION

Dr. Anthony H. Cincotta, the founder of Veroscience and the developer of Cycloset, teaches of the dopamine agonist bromocriptial and the effects on diabetes type 2. These issued US patents are: U.S. Pat. Nos. 5,468,755; 5,679,685; 5,756,513; 5,866,584; 6,004,972; 6,071,914; 6,075,020; 6,202,066 and 6,855,707. Published US patent applications that offer further teachings are: 20080200453; 20090137598; 20090137599; and 20090143390.

Relative to vitamin D and metabolism, Dr. Hector DeLuca recognizes and teaches of multiple vitamin D analogs, receptors, and modulating peripheral vitamin D receptors in bone to prevent or treat osteoporosis; but none of these teachings are applicable to central (brain) neurotransmitters. In U.S. Pat. No. 6,034,075 “Method of Treating Polycystic Ovarian Syndrome,” Dr. Susan Ihgs-Jacobs retales using vitamin D and calcium to induce ovulation. Polycystic Ovarian Syndrome is related to insulin resistance, and while metformin is effective in the clinical therapy for both endocrine dysfunction, gynecologic endocrinologists do not yet understand the relationships.

### PRIOR ART—BACKGROUND

### Bromocriptine (Parlodel 2.5 mg)

Bromocriptine is a dopamine agonist that has been successfully used for over twenty years in the treatment of infertility. A woman with infertility and amenorrhea, the absence of regular periods, is initially evaluated with a serum prolactin determination and a serum thyroid stimulation hormone (TSH) determination. If the serum thyroid stimulating hormone (TSH) is elevated, thyroid replacement is initiated. The thyroid stimulating releasing factor (TSRF) in the hypothalamic-pituitary axis can act as a stimulator of prolactin production. The two compounds that can inhibit prolactin production are prolactin inhibiting factor (PIF) and dopamine. More commonly, infertility and amenorrhea are caused by excessive pituitary production of prolactin. Prolactin can be excessively produced in these situations: inadequate prolactin inhibiting factor (PIF) from the hypothalamus; a pituitary prolactinoma, a prolactin producing tumorus adenoma; or in breastfeeding. In all these situations the elevated prolactin prevents ovulation, the release of a mature ova from the ovary; the etiology of the infertility and amenorrhea. Bromocriptine as a dopamine agonist acts like the prolactin inhibiting factor in women with elevated prolactin levels, by acting to prevent the production and secretion of the pituitary prolactin. Bromocriptine can also be employed to prevent post-partum breast engorgement for women who chose not to breastfeed.

“Hyperprolactinemia is the most common endocrine disorder of the hypothalamic-pituitary axis,” observed doctors Mah and Webster in their published review ‘Hyperprolactinemia: etiology, diagnosis, and management’ in Seminars in Reproductive Medicine. 2002 November; 20(4): 365-74. Doctors Mah and Webster continue and comment that, “a prolactinoma is the most common cause of chronic hyperprolactinemia, once pregnancy, primary hypothyroidism, and drugs that elevate serum prolactin have been excluded. A prolactinoma develops because of inadequate prolactin inhibiting factor (PIF) release from the hypothalamus. All of the neuroendocrine hormones from the hypothalamus that control pituitary hormone production and release are stimulating release factors with the exception of prolactin inhibiting factor (PIF). This is why hyperprolactinemia is the most common endocrine disorder of the hypothalamic-pituitary axis. Bromocriptine is the first-line therapy for hyperprolactinemia, and has been for over twenty years!

### BACKGROUND FOR THE PRESENT INVENTION

### Metabolism and Sunlight

Two factors are important relative to sunlight's ability to regulate metabolism: the circadian rhythm modulating early morning bright sunlight in the pineal-hypothalamus-pituitary axis where dopamine regulates peripheral metabolism via prolactin secretion or inhibition; and the total ultraviolet irradiation from sunrise to sunset, that regulates the vitamin D₁ and D₂ conversion to D₃. Therefore, there are at least two independent, sunlight-related, mechanisms to distinguish between summer metabolism and winter metabolism.

Other mechanisms that are also survival related but really not thought to be sunlight related influence metabolism, especially winter metabolism. These are: lepin, a hormone produced by fat cells; and ghrelin, a hormone produced by the gastric mucosal cells. Therefore, there are at least four separate physiological systems to regulate systemic metabolism and ensure survival, which are especially important during the winter months. The interactions of the multiple metabolic survival systems render scientific cause and effect relationships of a single system difficult to evaluate.

In terms of effect on insulin resistance, the metabolic syndrome and diabetes type 2, benefit can be anticipated by concurrently impacting both of the sunlight-related metabolic regulatory systems. Therefore, a therapy to modulate the circadian rhythm with a dopamine precursor and dopamine-like neurotransmitter, vitamin D₃, is herein described.

### Circadian Rhythm, Dopamine & Bromocriptine

Circadian rhythm is controlled by both the early morning light and the duration of daylight. The circadian rhythm is controlled via the effects of that light on the pineal gland as well as the hypophyseal-pituitary axis. The effects associated with decreased light exposure and the pineal gland functioning are well known; with the primary result being a reduction in the release of melatonin. However recent focus has been given to investigating the other half of the equation, the hypothalamic-pituitary axis. Many studies have been performed that now link dopamine, an important neurotransmitter, to weight gain and the metabolic syndrome that are associated with a decrease in seasonal sunlight:

“Compensatory weight gain due to dopaminergic hypofunction: new evidence and own incidental observations.” Reinholz J et al. Nutr Metab (Lond). 2008 December 1; 5:35.
Dr. Reinholz and colleagues reported that their; “findings support the crucial role of dopaminergic signaling for eating behaviors and offer an explanation for weight-gain after cessation of activities associated with high dopaminergic signaling. They further support the possibility that dopaminergic medication could be used to moderate food intake.”
“Deficits of mesolimbic dopamine neurotransmission in rat dietary obesity.” Geiger B M et al. Neuroscience. 2009 Apr. 10; 159(4):1193-9.
   Dr. Geiger and colleagues report that; “results demonstrate that deficits in mesolimbic dopamine neurotransmission are linked to dietary obesity. Depressed dopamine release may lead obese animals to compensate by eating palatable “comfort” food, a stimulus that released dopamine when laboratory chow failed.”

The dopamine agonist bromocriptine has been used for years to treat hyperprolactinaemia, a condition associated with abnormally high levels of the hormone prolactin. It is usually caused by diseases of the anterior pituitary gland as well as the hypothalamus, especially tumors in the pituitary known as prolactinomas. Dopamine agonists such as bromocriptine are the main avenue of treatment, since dopamine acts as an inhibiting factor for the release of prolactin. Treatment of hyperprolactinaemia with bromocriptine also however resulted in the discovery that the dopamine agonist has additional effects on body weight and composition.

Many studies have been performed to display the links between bromocriptine, dopamine, and obesity:
“Dopaminergic tone and obesity: an insight from prolactinomas treated with bromocriptine.” Doknic M et al. Eur J Endocrinol. 2002 July; 147(1):77-84.
   Dr. Doknic and colleagues report that; “It has recently been shown that increased body weight is associated with prolactinomas and that weight loss occurs with normalization of prolactin levels. On the other hand, decreased dopaminergic tone in humans is well correlated with obesity. The objective of this study was to correlate changes in prolactin levels with leptin and body mass index (BM) in patients with prolactinomas treated with the long-acting dopamine agonist bromocriptine (BC).”
   They concluded that; “The long-acting dopamine agonist BC, by increasing dopaminergic tone, may influence body weight and likely body composition by mechanisms in addition to reducing hyperprolactinemia in patients with prolactinomas.”
“Bromocriptine administration reduces hyperphagia and adiposity and differentially affects dopamine D2 receptor and transporter binding in leptin-receptor-deficient Zucker rats and rats with diet-induced obesity.” Davis L M et al. Neuroendocrinology. 2009; 89(2):152-62.
   Dr. Davis and colleagues report that; “The dopamine (DA) D(2) receptor (D2R) agonist bromocriptine (BC) decreases body fat in animal and human models and increases lean muscle mass, improves glucose intolerance and insulin resistance, and reduces triglycerides and free fatty acids. We have previously shown a negative correlation between D2R and body weight in obese individuals and in rodents . . . .”
   They conclude that; “These observations are all consistent with the existence of unique leptin-DA interactions and the hypothesis that there is hyposensitivity of the DA system in obesity.”
“Bromocriptine (Ergoset) reduces body weight and improves glucose tolerance in obese subjects.” Cincotta A H, Meier A H. Diabetes Care. 1996 June; 19(6):667-70.
   This describes; “A double-blind placebo controlled study investigated long-term effects of Ergoset, a new quick release formulation of bromocriptine, on body weight, body fat, and glucose tolerance in a group (n=17) of obese subjects who were instructed to follow a moderate hypocaloric diet.”
   Dr. Cincotta concludes that; “When combined with instruction to follow a moderate hypocaloric diet, Ergoset, but not placebo, improves glucose tolerance and promotes significant weight and body fat loss in obese subjects over an 18-week treatment period.”

Along with weight loss effects and prolactin inhibiting actions, bromocriptine has also been found to have positive treatment effects on those conditions associated with the metabolic syndrome as well as type 2 diabetes. The metabolic syndrome is a pre-diabetic state associated with: insulin resistance, increased weight, dyslipidemia, and hypertension.
“Activation of dopamine D2 receptors simultaneously ameliorates various metabolic features of obese women.” Kok P et al. Am J Physiol Endocrinol Metab. 2006 November; 291(5):E1038-43.
   Dr. Kok and colleagues report; “The metabolic syndrome comprises a cluster of metabolic anomalies including insulin resistance, abdominal obesity, dyslipidemia, and hypertension. Previous studies suggest that impaired dopamine D2 receptor (D2R) signaling is involved in its pathogenesis.”
   Their “results imply that short-term bromocriptine treatment ameliorates various components of the metabolic syndrome while it shifts energy balance away from lipogenesis in obese humans.”
“Modulation of monoaminergic neural circuits: potential for the treatment of type 2 diabetes mellitus.” Pijl H and Edo AM. Treatments of Endocrinology. 2002; 1(2): 71-8
   Dr. Pijl reports that, “Bromocriptine favorably affects glucose metabolism and various other components of the metabolic syndrome simultaneously to ameliorate the risk of damage to eyes, neural tissue, kidneys and the cardiovascular system in patients with type 2 diabetes mellitus.”
“Bromocriptine: a novel approach to the treatment of type 2 diabetes.” Pijl H. et al. Diabetes Care. 2000 August; 23(8): 1154-61
   Dr. Pijl reports, “In vertebrates, body fat stores and insulin action are controlled by the temporal interaction of circadian neuroendocrine oscillations. Bromocriptine modulates neurotransmitter action in the brain and has been shown to improve glucose tolerance and insulin resistance in animal models of obesity and diabetes.”
   From this report of the first human clinical trial of bromocriptine on 22 obese/diabetic patients (2000), Dr. Pijl concluded that; “bromocriptine improves glycemic control and glucose tolerance in obese type 2 diabetic patients.”
“Bromocriptine—unique formulation of a dopamine agonist for the treatment of type 2 diabetes.” Scranton R Cincotta A. Expert Opin Pharmacother, 2010 February; 11(2):269-79
   Dr. Scranton reports in the abstract that the “Take Home Message” is, “Bromocriptine-QR is indicated to be used alone or in conjunction with all available treatments for type 2 diabetes. Although the mechanism of action is not fully understood, bromocriptine-QR's action points to a central target in the brain (hypothalamus) which may explain the -observed peripheral improvements in metabolic parameters.”

Following the discovery and investigation of the positive effects of bromocriptine on the metabolic syndrome and type 2 diabetes; additional research has been performed to make the link between the aforementioned conditions, dopaminergic modulation, and circadian rhythm.
“Association of the anti-diabetic effects of bromocriptine with a shift in the daily rhythm of monoamine metabolism within the suprachiasmatic nuclei of Syrian hamster.”
Luo, S. et al. Chronobiotics Investigations. 2000 March; 17(2): 155-72
   Dr. Luo states that, “bromocriptine, a dopamine D2 agonist, inhibits seasonal fattening and improves seasonal insulin resistance in Syrian hamsters. Bromocriptine-induced resetting of daily patterns of suprachiasmatic nuclei neurotransmitter metabolism is associated with the effects of bromocriptine on attenuation of the obese insulin-resistant and glucose-intolerant condition.”

### Cycloset (Bromocriptine 0.8 mg)

Cycloset is the first-in-class drug of circadian rhythm chronobiotic modulator, targeting the hypothalamus-pituitary axis to control and dictate systemic cellular metabolism. Cycloset, a dopamine agonist, mimics early morning light to reset the circadian rhythm to summer metabolism from the winter metabolism of insulin resistance, diabetes type 2, and the metabolic syndrome.

The circadian rhythm is controlled by both the early morning light and the duration of daylight. The circadian rhythm is controlled via the effects of that light on the pineal gland and the hypophyseal-pituitary axis. In a separate news report of the FDA approval of Cycloset, Jennifer Boggs of BioWorld Today reported that; “It was a big win for Tiverton, R. I.-based Veroscience, which has operated quietly since its founding in 2001. But work on Cycloset goes back much farther, having been started 28 years ago by the company's founder and president, Anthony Cincotta, who first began working on the program as a graduate student at Louisiana State University.”

The article further goes on; “Animals entering annual states of hibernation or migration become obese and insulin-resistant—especially prediabetic—yet as they move into the next season's cycle, these conditions vanish with no lingering effects, indicating an “endogenous clock in the brain [that regulates] neurochemistry changes as they go throughout the year, [Cincotta] said. Cincotta and his team mapped out a blueprint of animals' summer and winter cycles and found that the “24-hour activity of dopamine varied between winter animals and summer animals.” Further research confirmed that the presence or absence of dopamine could produce or reverse the animals' prediabetic conditions, and similar results were observed in human trials, [Cincotta} said. Cycloset is designed as a once-daily, quick-release product, to be administered in the morning, as a “single pulse to the body to provide that circadian peak at that time,” Cincotta said. “The idea is to copy the way nature returns prediabetic to normal.” In clinical testing, Cycloset clearly improved glycemic control. When taken in the morning, patients showed improved postprandial glucose without increased plasma insulin concentrations and demonstrated beneficial effects on postmeal glycemic control hours after the drug has been cleared from circulation.”

On May 6, 2009, the US FDA approved Cycloset for the treatment of type 2 diabetes. This is a first-in-class use of a circadian rhythm modulator that effectively treats diabetes type 2. This is the first-in-class use of a dopamine agonist utilized to effectively treat diabetes type 2. This is the first-in-class use of a central nervous system neurotransmitter utilized to effectively treat a systemic cellular disease—insulin resistance, the etiology of diabetes type 2.

The Veroscience press release of May 6, 2009 states; “Cycloset improves glycemic control across a broad patient population as a monotherapy, as an adjunctive therapy to sulfonylurea, metformin plus sulfonylurea, and single or dual oral hypoglycemic agent therapies.”

The full press release by VeroScience for the FDA-approval of Cycloset read as follows:
“TIVERTON, R. I. & BRISTOL, Tenn.—(BUSINESS WIRE)—May 6, 2009—VeroScience, in collaboration with S2 Therapeutics, Inc. (S2), announced today that the U.S. Food and Drug Administration (FDA) approved its first-in-class drug Cycloset for the treatment of Type 2 diabetes. Cycloset improves glycemic control across a broad patient population as a monotherapy or as an adjunctive therapy to sulfonylurea, metformin plus sulfonylurea, and single or dual oral hypoglycaemic agent therapies.
Cycloset is the first drug to be approved subsequent to the FDA's new guidelines that require studies demonstrating that diabetes drugs do not increase cardiovascular risk. A 52 week, double-blind safety trial of 3,000 patients treated with Cycloset did not show an increase in pre-specified and independently adjudicated adverse cardiovascular outcomes—a composite of myocardial infarction, stroke, hospitalization for unstable angina, congestive heart failure, and revascularization surgery—compared to patients taking a placebo (HR 0.58; CI 0.35-0.96).
“For patients newly diagnosed with Type 2 diabetes or those who cannot adequately control their blood sugar with currently available medications, Cycloset provides a completely new approach to treating diabetes,” said J. Michael Gaziano, MD., Cardiologist, Associate Professor, Division of Aging, Brigham & Women's Hospital and principal investigator of the Cycloset Safety Trial. “In addition, patients with Type 2 diabetes are at high-risk for cardiovascular events, so it's important that Cycloset has been demonstrated not to increase the risk of cardiovascular events such as heart attacks, and may actually have potential to lower this risk.”
Cycloset represents a new therapeutic approach in the management of Type 2 diabetes. It is the first drug for patients with diabetes that targets the body's dopamine activity, a chemical messenger between neurons, or nerve cells, within the nervous system. A dopamine agonist, Cycloset increases. dopamine activity. While the specific mechanism by which Cycloset improves glycemic control in humans is unknown, the development of Cycloset for the treatment of Type 2 diabetes was based upon preclinical studies that have shown brain dopamine activity to be low in metabolic disease states and that this factor contributes to multiple metabolic dysfunctions such as insulin resistance—the loss of the body's ability to respond to the blood sugar lowering effects of insulin—observed in Type 2 diabetes. Moreover, preclinical studies of diabetic animals have shown that treatment with a dopamine agonist as in Cycloset acts upon the central nervous system to reset and improve control of peripheral metabolism.
“This approval is a major milestone for. VeroScience and also represents the culmination of many years of intense research and development activities. We're very pleased that these prodigious efforts will now translate to provide patients with a new approach to treating Type 2 diabetes,” said Anthony H. Cincotta, Ph.D., President and Chief Scientific Officer, VeroScience.
Charles P. Sutphin, President and CEO of S2 Therapeutics, Inc. said, “We are extremely happy to have been able to support and work with VeroScience to bring this diabetes therapy to the U.S. market. With the United States approval, we will collectively initiate efforts to complete applications globally with an immediate focus in Europe and Canada.” S2 Co-chairman and co-founder Brian Schrader said “S2 Therapeutics and VeroScience intend to seek a partner to commercialize Cycloset in order to bring this important new therapy to patients as soon as possible.”

About Cycloset and the Biological Clock
Preclinical studies indicate that while an increase in dopamine activity leads to improvements in diabetes, the time of day of the increased dopamine activity is also important. Studies in diabetic animals have shown that increased dopaminergic activity at a particular time of day is most effective in “resetting” the biological clock neurochemistry to a physiology that improves diabetic dysmetabolism. Taken orally, once-a-day, in the morning, Cycloset provides a single brief pulse of dopamine agonist activity shortly after its administration. Morning Cycloset improves post-prandial glucose without increasing plasma insulin concentrations and the beneficial effects of Cycloset on post-meal glycemic control in patients with Type 2 diabetes are demonstrable many hours after the drug has been substantially cleared from the circulation, for example at lunch and dinner.
The Cycloset Safety Trial, a 3,000 patient, one-year study, demonstrated that Cycloset at doses up to 4.8 mg per day used to treat Type 2 diabetes was not different from placebo regarding the rate of occurrence of all-cause serious adverse events. None of the serious adverse events grouped by System-Organ-Class occurred more than 0.3 percentage points higher with Cycloset than with placebo. Cycloset can cause hypotension, including orthostatic hypotension, and syncope, particularly upon dose initiation or escalation. The primary reason for discontinuation from clinical trials of Cycloset was nausea, which was mild to moderate and transient during the beginning of therapy.
“Cycloset represents a new treatment paradigm for Type 2 diabetes. The clinical development of Cycloset involved the collection of a vast amount of safety data prior to the drug's approval. The information compiled to date has demonstrated that this novel therapy is both safe and effective in treating Type 2 diabetes alone or in combination with other drugs used to treat Type 2 diabetes,” said Richard Scranton M.D., M.P.H., Chief Medical Officer, VeroScience.”

Cycloset is important due to the fact that for the first time, a dopamine modulating drug has been FDA-approved for the treatment of metabolic disorders associated with insulin-resistance that have a primary mode of action to modulate the circadian rhythm (chronobiotic). Though not approved specifically for the treatment of obesity, evidence clearly supports the weight loss associated with dopamine modulation.

### Caffeine Priming of the Hypothalamic Dopamine Receptor

Caffeine is a neurostimulant, and the most widely used drug in the world. The actions as a neurostimulant are both general, such as increased alertness and anti-fatigue; and specific, such as targeted effects on certain anatomic regions of the brain and receptors.

Caffeine, in therapeutic doses, has been reported to sensitize the dopamine receptors in the hypothalamus of the brain:
“The role of dopamine in the behavioral effects of caffeine in animals and humans.”
Garrett B E, Griffiths R R. Pharmacol Biochem Behay. 1997 July; 57(3):533-41
   Dr. Garrett reports; “Recently, caffeine has been shown to enhance dopaminergic activity, presumably by competitive antagonism at adenosine receptors that are colocalized and interact functionally with dopamine receptors. Thus, caffeine, as a competitive antagonist at adenosine receptors, may produce its behavioral effects by removing the negative modulatory effects of adenosine from dopamine receptors, thus stimulating dopaminergic activity.”
“Prior Treatment (Priming) with Caffeine Sensitizes D2-Dopamine-Mediated Contralateral Rotational Behavior in 6-Hydroxydopamine-Lesioned Rats.”
Pollack A E et al. Pharmacology. 2010; 86(2):73-78.
   Following this study, Dr. Pollack and colleagues conclude that; “These results suggest that prior administration of caffeine can sensitize D2 dopamine-mediated rotational behavior in dopamine-depleted rats.”
“Dopaminergic effects of caffeine in the human striatum and thalamus.”
Kassinen V et al. Neuroreport. 2004 Feb. 9; 15(2):281-5.
   Dr. Kassinen and colleagues report that; “Compared to oral placebo, 200 mg oral caffeine induced a 12% decrease in midline thalamic binding potential (p<0.001). A trend-level increase in ventral striatal [(11)C]raclopride binding potential was seen with a correlation between caffeine-related arousal and putaminal dopamine D(2) receptor binding (r=−0.81, p=0.03). The findings indicate that caffeine has effects on dopaminergic neurotransmission in the human brain, which may be differential in the striatum and the thalamus.”

By employing caffeine as a hypothalamic priming and sensitizing neurostimulant in conjunction with a dopamine precursor N-acetyl-L-tyrosine or a dopamine agonist such as bromocriptine, and vitamin D₃, a more profound effect can be expected in dopaminergic agonist activity by the combination of all three substances dosed concurrently early in the morning. Therefore by using caffeine as a priming agent, the doses of either a dopamine precursor such as N-acetyl-L-tyrosine or a dopamine agonist such as bromocriptine and vitamin D₃ can remain within the safe therapeutic range, and still act to produce a profound effect on both dopamine and prolactin to modulate the circadian rhythm.

### Winter Metabolism: Vitamin D Deficiency

Dr. Yvonne Frost reported in 2009 that; “A fall in vitamin D in the form of circulating calcidiol is the stimulus for the winter response, which consists of an accumulation of fatness (obesity) and the induction of a winter metabolism (the metabolic syndrome). Vitamin D deficiency can account for the secular trends in the prevelance of obesity and for individual differences in its onset and severity. It may be possible to reverse the increasing prevelance of obesity by improving vitamin D status.” This was reported in Medical Hypothesis. 2009 March; 72(3): 314-21 in the article, “Vitamin D deficiency is the cause of common obesity.”

Vitamin D is really a prohormone and a hormone. Vitamin D is a fat-soluble prohormone that is dependent upon ultraviolet radiation from sunlight to convert it to its active hormone, 25-hydroxyvitamin D₃ (vitamin D₃). This conversion into the active vitamin D₃ occurs in the sun-exposed peripheral skin. The increased time as well as intensity of sun exposure, and the more skin exposed, results in greater amounts of vitamin D₃ produced by ultraviolet irradiation of the sun. Only ultraviolet light can convert the prohormone vitamin D to its active form of vitamin D₃ within the skin.

Numerous medical references document the peripheral influence of adequate vitamin D₃ on summer metabolism relative to all components of the metabolic syndrome and related to inadequate vitamin D₃ and winter metabolism:
“Vitamin D deficiency is the cause of common obesity.”
Foss Y J. Med Hypotheses. 2009 March; 72(3):314-21.
   Foss concludes that; “It is here proposed that a fall in vitamin D in the form of circulating calcidiol is the stimulus for the winter response, which consists of an accumulation of fat mass (obesity) and the induction of a winter metabolism (the metabolic syndrome). Vitamin D deficiency can account for the secular trends in the prevalence of obesity and for individual differences in its onset and severity. It may be possible to reverse the increasing prevalence of obesity by improving vitamin D status.”
“Relationships of low serum vitamin D3 with anthropometry and markers of the metabolic syndrome and diabetes in overweight and obesity.”
McGill et al. Nutr J 2008 Jan. 28; 7:4.
   McGill and colleagues report that; “Low serum 25 hydroxyvitamin D3 (vitamin D3) is known to perturb cellular function in many tissues, including the endocrine pancreas, which are involved in obesity and type II diabetes mellitus (TIIDM). Vitamin D3 insufficiency has been linked to obesity, whether obesity is assessed by body mass index (BMI) or waist circumference (waist). Central obesity, using waist as the surrogate, is associated with the metabolic syndrome (MetSyn), insulin resistance, TIIDM and atherosclerotic cardiovascular disease (CVD).”
“Hypovitaminosis D in obese children and adolescents: relationship with adiposity, insulin sensitivity, ethnicity, and season.” Alemzadeh R et al. Metabolism, 2008 February; 57(2):183-91
   Alemzadeh and colleagues report that; “serum 25(OH) D was positively correlated with insulin sensitivity, which was FM mediated, but negatively correlated with HbA(1c), implying that obese children and adolescents with low vitamin D status may be at increased risk of developing impaired glucose metabolism independent of body adiposity. Additional studies are needed to evaluate the underlying mechanisms.”
“Vitamin D deficiency and the risk of incident Type 2 diabetes.” Michos E D. Furture Cardiol. 2009 January; 5(1): 15-8
   Michos reports that; “Cross-sectional studies have demonstrated that lower serum 25-hydroxyvitamin D (25[OH]D) levels are associated with obesity, the metabolic syndrome, impaired glucose tolerance and diabetes . . . . The article by Knekt et al. was the first prospective study to demonstrate that low 25(OH)D levels predict incident diabetes . . . . While animal studies and smaller interventional trials in humans suggest that vitamin D supplementation improves measures of insulin sensitivity and glucose tolerance, larger interventional trials are warranted to determine if vitamin D treatment at adequate doses can prevent diabetes.”
“The effect of vitamin D3 on insulin secretion and peripheral insulin sensitivity in type 2 diabetic patients.” Borissova A M et al. Int J Clin Pract 2003 May; 57(4): 258-61
   Borissova and colleagues state; “The aim of this study was to evaluate the effect of vitamin D3 supplementation on insulin secretion and insulin resistance . . . . Bearing in mind that the main defects in type 2 diabetes mellitus are reduced FPIS and insulin resistance, and the favourable effect vitamin D3 had on them, we suggest vitamin D3 deficiency may at least partly contribute to the impairment of insulin secretion and probably of insulin action. Our results suggest that vitamin D3 supplementation could be an element in the complex treatment of type 2 diabetes mellitus during the winter.”
“Role of vitamin D in the pathogenesis of type 2 diabetes mellitus.” Palomer X et al. Diabetes Obes Metab, 2008 March; 10(3): 185-97
   Palomer and colleagues report that; “Vitamin D deficiency has been shown to alter insulin synthesis and secretion in both humans and animal models. It has been reported that vitamin D deficiency may predispose to glucose intolerance, altered insulin secretion and type 2 diabetes mellitus. Vitamin D replenishment improves glycaemia and insulin secretion in patients with type 2 diabetes with established hypovitaminosis D, thereby suggesting a role for vitamin D in the pathogenesis of type 2 diabetes mellitus.”

All of these cited references attribute insulin resistance and the metabolic syndrome to a single factor, vitamin D deficiency. These cited references do not postulate if the mechanism of action of vitamin D deficiency and insulin resistance, as being peripherally functional in each individual cell, or as being centrally controlled by the hypothalamic-pituitary axis (like Cycloset).

Although it is likely that both mechanisms are present, a recent medical imaging study noted the presence of vitamin D₃ receptors in the hypothalamus of the brain in tissue when performing immunohistochemical staining. This vitamin D₃ receptor was presumed to be in the dopaminergic neurons.
“Distribution of the vitamin D receptor and 1 alpha-hydroxylase in human brain.”
Eyles D W et al. J Chem Neuroanat. 2005 January; 29(1):21-30
   “This paper reports, for the first time, the distribution of the 1,25-dihydroxyvitamin D3 receptor (VDR), and 1alpha-hydroxylase (1alpha-OHase), the enzyme responsible for the formation of the active vitamin in the human brain . . . . The strongest immunohistochemical staining for both the receptor and enzyme was in the hypothalamus and in the large (presumably dopaminergic) neurons within the substantia nigra.”
“Distribution of vitamin D binding protein expressing neurons in the rat hypothalamus.”
Jirikowski G F et al. Histochem Cell Biol, 2009 March; 131(3):365-70
   Jirikowski and colleagues; “observed immunostaining for vitamin D binding protein (DBP) in rat hypothalamus . . . DBP was also observed in widespread axonal projections throughout the lateral hypothalamus, the median eminence and the posterior pituitary lobe . . . DBP may be synthesized and transported along with the classical neurohypophyseal hormones. The multiple locations of DBP-expressing neurons indicate multiple functional properties: DBP may be released from in the posterior lobe, it may act as a hypophyseotropic factor and as a central neuroactive substance.”
   Jirikowski concludes the vitamin D₃ can be synthesized and transported in the hypothalamus-pituitary axis, like dopamine. Vitamin D₃ as a classical neurotransmitter, is currently under investigation by multiple groups of neuroendocrinologists.

### THE DESCRIPTION OF THE PRESENT INVENTION

The present invention is intended to modulate the circadian rhythm to address the winter metabolism of insulin resistance and the metabolic syndrome. The present invention consists of five components:
1. The administration (oral ingestion) of a dopamine precursor, L-Tyrosine, to evoke the timed pulsitile production of dopamine in the hypothalamus
2. The use of N-acetyl-L-tyrosine, a very water soluble configuration of the L-tyrosine, to evoke the pulsitile dopamine production
3. Caffeine priming of the dopamine receptors in the hypothalamus of the brain
4. The concurrent administration of vitamin D₃, the physiologically active vitamin D, that does not rely upon sunlight, with the dopamine precursor N-acetyl-L-tyrosine or the dopamine agonist bromocriptine to reset and modulate the circadian rhythm
5. The early morning, once per day, administration of the dopamine precursor N-acetyl-L-tyrosine or the dopamine agonist bromocritpine, caffeine and vitamin D₃ to evoke a pulsitile hypothalamic neurotransmitter response and reset and modulate the circadian rhythm
   Each component of the present invention will be explained more in detail:

1. The administration (oral ingestion) of a dopamine precursor, L-tyrosine, to evoke the timed pulsitile production of dopamine in the hypothalamus

The biosynthetic pathway that produces dopamine has three successive precursors: phenylalanine, L-tyrosine (4-hydroxyphenylalanine), and L-DOPA (3,4-dihydro-L-phenylalanine).

Phenylalanine is supplied via the diet, absorbed from the small intestines, and circulated to the liver. In the liver, phenylalanine is converted to L-tyrosine. (L-tyrosine can also be diet supplied.) L-tyrosine is then released from the liver and enters the systemic blood circulation. L-tyrosine is converted to L-DOPA in specific tissues, like certain brain tissues and the adrenal tissues. In certain brain tissues, L-DOPA is converted into the catecholamines dopamine, epinephrine, and norepinephrine. In the thyroid gland L-tyrosine is converted into the thyroid hormones triodothyronine (T₃) and thyroxine (T₄). L-tyrosine is also the precursor of melanin.

The serum level of L-tyrosine is the rate-limiting factor for stimulation of the production of L-DOPA and dopamine in the brain tissues. Therefore, the increased serum level of L-tyrosine allows for the increased production and release of the neurotransmitter dopamine. This is accomplished by the enzyme tyrosine hydroxylase.
a) “Tyrosine hydroxylase (TH) catalyzes the conversion of L: -tyrosine to L: -dopa, which is the initial and rate-limiting step in the biosynthesis of catecholamines [CA; dopamine (DA), noradrenaline, and adrenaline], and plays a central role in the neurotransmission and hormonal actions of CA.”
Nakashima, A et al. “Role of N-terminus of tyrosine hydroxylase in the biosynthesis of catecholamines. ” J Neural Transm., 2009 Apr. 25.
b) “TH is a tetrahydrobiopterin-requiring, iron-containing monooxygenase. It catalyses the conversion of L-tyrosine to L-dopa, which is the first, rate-limiting step in the biosynthesis of catecholamines (dopamine, noradrenaline and adrenaline), the central and sympathetic neurotransmitters and adrenomedullary hormones.”
Nagatsu, T. “Tyrosine hydroxylase: human isoforms, structure and regulation in physiology and pathology.” Essays Biochem., 1995; 30:15-35
   Although L-DOPA is the intermediate substrate between L-tyrosine and dopamine, L-DOPA administration is avoided because of systemic adverse events of:
Hypotension
Arrhythmias
Nausea
Anxiety
Confusion
2. The use of N-acetyl-L-tyrosine, a very water soluble configuration of the L-tyrosine, to evoke the pulsitile dopamine production.
   L-tyrosine is partially water soluble. N-acetyl-L-tyrosine is very water soluble. Since the intent of this invention is to supply a pulsitile dopamine precursor to stimulate dopamine synthesis and release within the hypothalamus-pituitary axis, the N-acetyl-L-tyrosine is employed because of the optimal bowel absorption due to its high water solubility. The high solubility index of the N-acetyl-L-tyrosine also allows rapid systemic transport to the hypothalamus and rapid absorption at the hypothalamus.
3. Caffeine priming of the dopamine receptors in the hypothalamus of the brain
   Caffeine is a well-known and highly consumed neurostimulant. Within the brain, caffeine acts as a competitive inhibitor of the adenosine receptor, thus blocking the inhibitory action of adenosine. This action of inhibiting the adenosine receptor results in the stimulation of dopaminergic activity.
4. The concurrent administration/ingestion of vitamin D3, the physiologically active vitamin D, that does not rely upon sunlight, with the dopamine precursor N-acetyl-L-tyrosine or the dopamine agonist bromocriptine to reset and modulate the circadian rhythm.
   The active oral vitamin D₃, both a hormone and a neurotransmitter, is concurrently administered/ingested with either N-acetyl-L-tyrosine or bromocriptine to provide two dopamine agonist-like entities to the hypothalamus to modulate the circadian rhythm. The modulation of the circadian rhythm is intended to correct or prevent the winter metabolism and the metabolic syndrome.
5. The early morning, once per day, administration/ingestion of the dopamine precursor N-acetyl-L-tyrosine or the dopamine agonist bromocriptine, caffeine and vitamin D3 to evoke a pulsitile hypothalamic neurotransmitter response and reset and modulate the circadian rhythm
   This is to evoke a Cycloset-like response by the once-a-day, early morning administration/ingestion of the dopamine neurotransmitter precursor or a dopamine agonist, receptor priming agent, and the active hormone, neurotransmitter vitamin D₃. The Cycloset-like response is to modulate the circadian rhythm into a year-round summer metabolism. This modulation of the circadian rhythm is to prevent or correct the metabolic syndrome of winter metabolism.

### Proposed Dosages:

| | |
|---|---|
| 1. N-acetyl-L-tyrosine | |
| 200-1500 mg range | Best Mode: 500 mg |
| 2. Caffeine | |
| 100-200 mg range | Best Mode: 100 mg |
| 3. Vitamin D₃ | |
| 200-1200 IU range | Best Mode: 1200 mg |

According to the CDC, the half-life of vitamin D₃ is 1-2 days. Vitamin D toxicity has been reported with doses of vitamin D₁ & D₂ of 2000 IU/day or more. Further vitamin D toxicity information can be found in the Merck manual Professional, which reports that; “In adults, taking 1250 μg (50,000 IU)/day for several months can produce toxicity.” The half-life of vitamin D₁ & D₂ is 2-7 days and stored in body fat and the liver.

The invention thus comprises a method to modulate the human circadian rhythm to effect summer metabolism, comprising: administering a dosage of N-acetyl-L-tyrosine, caffeine and vitamin D₃ , wherein the dosage is taken orally, daily. The dosage of N-acetyl-L-tyrosine is preferably between 200 to 1500 mg. The dosage of caffeine is preferably between 100 to 200 mg. The dosage of vitamin D₃ is preferably between 200 to 1200 mg. The dosage of N-acetyl-L-tyrosine is preferably about 500 mg. The dosage of caffeine is preferably about 100 mg. The dosage of vitamin D₃ is preferably about 1200 mg.
The invention also comprises a method and formulation for the daily oral delivery of a dopamine agonist consisting of a single formulation of bromocriptine and vitamin D₃. The dose of vitamin D₃ consists of a dose preferably between 1000 IU and 2000 IU.

The invention also comprises a method and formulation for the daily oral delivery of a dopamine agonist comprised of a single formulation of: bromocriptine, caffeine and vitamin D₃ . The vitamin D₃ consists of a therapeutic dose of preferably between 1000 IU and 2000 IU. The caffeine consists of a therapeutic dose of preferably between 100 mg and 200 mg, and wherein the single formulation is preferably taken orally, in a morning consumption.

I claim:

1. A method to modulate the human circadian rhythm to effect summer metabolism, comprising:
administering a dosage of N-acetyl-L-tyrosine, caffeine and vitamin D₃, wherein the dosage is taken orally, daily.

2. The method as recited in claim 1, wherein the dosage of N-acetyl-L-tyrosine is between 200 to 1500 mg.

3. The method as recited in claim 1, wherein the dosage of caffeine is between 100 to 200 mg.

4. The method as recited in claim 1, wherein the dosage of vitamin D₃ is between 200 to 1200 mg.

5. The method as recited in claim 1, wherein the dosage of N-acetyl-L-tyrosine is about 500 mg.

6. The method as recited in claim 1, wherein the dosage of caffeine is about 100 mg.

7. The method as recited in claim 1, wherein the dosage of vitamin D₃ is about 1200 mg.

8. A method and formulation for the daily oral delivery of a dopamine agonist consisting of a single formulation of bromocriptine and vitamin D₃.

9. The method and formulation as recited in claim 8, wherein vitamin D₃ consists of a dose between 1000 IU and 2000 IU.

10. A method and formulation for the daily oral delivery of a dopamine agonist comprised of a single formulation of: bromocriptine, caffeine and vitamin D₃.

11. The method and formulation as recited in claim 10, wherein the vitamin D₃ consists of a therapeutic dose of between 1000 IU and 2000 IU.

12. The method and formulation as recited in claim 10, wherein the caffeine consists of a therapeutic dose of between 100 mg and 200 mg.

13. The method and formulation of claim 10, wherein the single formulation is taken orally, in a morning consumption.
